(19) United States
(12) Patent Application Publication
(10) Pub. No.: US 2019/0314313 A1
(43) Pub. Date: Oct. 17, 2019

(54) HOMEOSTATIC REGULATION OF L-DOPA BIOSYNTHESIS

(71) Applicant: BOARD OF REGENTS, THE UNIVERSITY OF TEXAS SYSTEM, Austin, TX (US)

(72) Inventors: Andrew D. Ellington, Austin, TX (US); Ross Thyer, Austin, TX (US)

(21) Appl. No.: 16/341,222

(22) PCT Filed: Oct. 11, 2017
(86) PCT No.: PCT/US17/56177

§ 371 Date: Apr. 11, 2019

Related U.S. Application Data
(60) Provisional application No. 62/406,559, filed on Oct. 11, 2016.

(51) Int. Cl. A61K 31/198; C12R 1/40; C12N 15/113; C12N 15/78; C12P 13/06

(52) U.S. Cl. CPC A61K 31/198; C12R 1/40; C12N 15/113; C12N 2800/101; C12P 13/06; C12Y 101/01049; C12N 2330/51; C12N 15/78

(57) ABSTRACT

Disclosed herein are methods and compositions for the production of L-3,4-dihydroxyphenylalanine from a bacteria.

### CROSS-REFERENCE TO RELATED APPLICATIONS

This application claims benefit of U.S. Provisional Application No. 62/406,559, filed Oct. 11, 2016, incorporated herein by reference in its entirety.

### BACKGROUND

L-DOPA, or L-3,4-dihydroxyphenylalanine, is a chemical that is made and used as part of the normal biology of humans, some animals and plants. Some animals and humans make it via biosynthesis from the amino acid L-tyrosine. L-DOPA is the precursor to the neurotransmitters dopamine, norepinephrine (noradrenaline), and epinephrine (adrenaline) collectively known as catecholamines. Furthermore, L-DOPA itself mediates neurotrophic factor release by the brain and

CNS. As a drug, it is used in the clinical treatment of Parkinson's disease and dopamine-responsive dystonia.

L-DOPA crosses the protective blood-brain barrier, whereas dopamine itself cannot. Thus, L-DOPA is used to increase dopamine concentrations in the treatment of Parkinson's disease and dopamine-responsive dystonia. Once L-DOPA has entered the central nervous system, it is converted into dopamine by the enzyme aromatic L-amino acid decarboxylase, also known as DOPA decarboxylase. Pyridoxal phosphate (vitamin B6) is a required cofactor in this reaction, and may occasionally be administered along with L-DOPA, usually in the form of pyridoxine.

What is needed in the art are methods of producing L-DOPA.

### SUMMARY

Disclosed herein is a genetically engineered cell capable of producing L-3,4-dihydroxyphenylalanine, wherein said cell is transformed with a gene encoding PP2551 of Pseudomonas putida. Also disclosed are cell lines capable of producing L-3,4-dihydroxyphenylalanine. Further disclosed are methods of producing L-3,4-dihydroxyphenylalanine, using a cell transformed with a gene encoding PP2551 of Pseudomonas putida.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows gene PP2551 (named DopA) from Pseudomonas putida. This gene is an L-DOPA responsive transcription factor.

FIG. 2 defines the minimal promoter for DopA.

FIG. 3 depicts a schematic of a circuit for L-DOPA production with homeostatic regulation. A genetic circuit for L-DOPA biosynthesis containing a positive feedback loop for homeostatic control of L-DOPA production is shown. Production of L-DOPA by HpaB (the product of the hpaB gene) activates the L-DOPA responsive transcription factor DopA, which is bound to a specific promoter sequence upstream of the hpaB gene. Activated DopA recruits bacterial transcriptional machinery to the promoter resulting in increased transcription of the hpaB gene. Increased transcription of the hpaB gene increases the amount of HpaB protein within the cell, in turn increasing the intracellular level of L-DOPA, resulting in a positive feedback signal. Bacterial cells eventually enter a steady-state phase of L-DOPA production without the need for external induction.

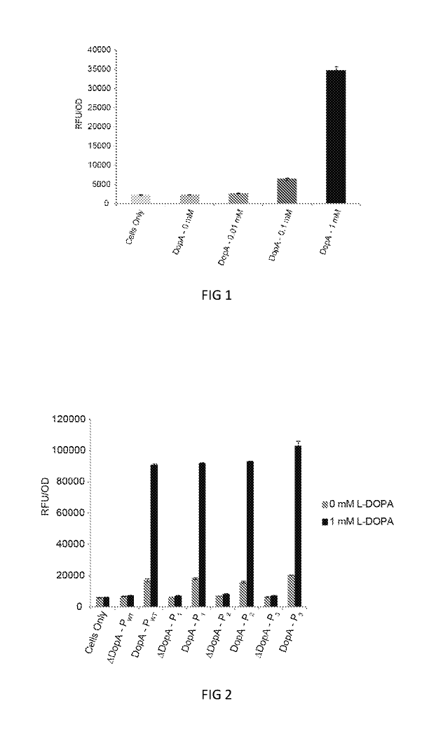

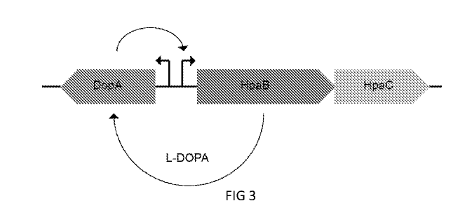

### DETAILED DESCRIPTION

### Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, reference to “an agent” includes mixture of two or more such agents, and the like.

“Optional” or “optionally” means that the subsequently described event or circumstance can or cannot occur, and that the description includes instances where the event or circumstance occurs and instances where it does not.

It is understood that throughout this specification the identifiers “first” and “second” are used solely to aid the reader in distinguishing the various components, features, or steps of the disclosed subject matter. The identifiers “first” and “second” are not intended to imply any particular order, amount, preference, or importance to the components or steps modified by these terms.

By convention, polynucleotides that are formed by 3′-5′ phosphodiester linkages (including naturally occurring polynucleotides) are said to have 5′-ends and 3′-ends because the nucleotide monomers that are incorporated into the polymer are joined in such a manner that the 5′ phosphate of one mononucleotide pentose ring is attached to the 3′ oxygen (hydroxyl) of its neighbor in one direction via the phosphodiester linkage. Thus, the 5′-end of a polynucleotide molecule generally has a free phosphate group at the 5′ position of the pentose ring of the nucleotide, while the 3′ end of the polynucleotide molecule has a free hydroxyl group at the 3′ position of the pentose ring. Within a polynucleotide molecule, a position that is oriented 5′ relative to another position is said to be located “upstream,” while a position that is 3′ to another position is said to be “downstream.” This terminology reflects the fact that polymerases proceed and extend a polynucleotide chain in a 5′ to 3′ fashion along the template strand. Unless denoted otherwise, whenever a polynucleotide sequence is represented, it will be understood that the nucleotides are in 5′ to 3′ orientation from left to right.

As used herein, it is not intended that the term “polynucleotide” be limited to naturally occurring polynucleotide structures, naturally occurring nucleotides sequences, naturally occurring backbones or naturally occurring internucleotide linkages. One familiar with the art knows well the wide variety of polynucleotide analogues, unnatural nucleotides, non-natural phosphodiester bond linkages and internucleotide analogs that find use with the invention.

As used herein, the expressions “nucleotide sequence,” “sequence of a polynucleotide,” “nucleic acid sequence,” “polynucleotide sequence”, and equivalent or similar phrases refer to the order of nucleotide monomers in the nucleotide polymer. By convention, a nucleotide sequence is typically written in the 5′ to 3′ direction. Unless otherwise indicated, a particular polynucleotide sequence of the invention optionally encompasses complementary sequences, in addition to the sequence explicitly indicated.

As used herein, the term “gene” generally refers to a combination of polynucleotide elements, that when operatively linked in either a native or recombinant manner, provide some product or function. The term “gene” is to be interpreted broadly, and can encompass mRNA, cDNA, cRNA and genomic DNA forms of a gene. In some uses, the term “gene” encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some aspects, genes do not encode a polypeptide, for example, ribosomal RNA genes (rRNA) and transfer RNA (tRNA) genes. In some aspects, the term “gene” includes not only the transcribed sequences, but in addition, also includes non-transcribed regions including upstream and downstream regulatory regions, enhancers and promoters. The term “gene” encompasses mRNA, cDNA and genomic forms of a gene.

In some aspects, the genomic form or genomic clone of a gene includes the sequences of the transcribed mRNA, as well as other non-transcribed sequences which lie outside of the transcript. The regulatory regions which lie outside the mRNA transcription unit are termed 5′ or 3′ flanking sequences. A functional genomic form of a gene typically contains regulatory elements necessary, and sometimes sufficient, for the regulation of transcription.

The term “promoter” is generally used to describe a DNA region, typically but not exclusively 5′ of the site of transcription initiation, sufficient to confer accurate transcription initiation. In some aspects, a “promoter” also includes other cis-acting regulatory elements that are necessary for strong or elevated levels of transcription, or confer inducible transcription. In some embodiments, a promoter is constitutively active, while in alternative embodiments, the promoter is conditionally active (e.g., where transcription is initiated only under certain physiological conditions).

Generally, the term “regulatory element” refers to any cis-acting genetic element that controls some aspect of the expression of nucleic acid sequences. In some uses, the term “promoter” comprises essentially the minimal sequences required to initiate transcription. In some uses, the term “promoter” includes the sequences to start transcription, and in addition, also include sequences that can upregulate or downregulate transcription, commonly termed “enhancer elements” and “repressor elements,” respectively.

Specific DNA regulatory elements, including promoters and enhancers, generally only function within a class of organisms. For example, regulatory elements from the bacterial genome generally do not function in eukaryotic organisms. However, regulatory elements from more closely related organisms frequently show cross functionality. For example, DNA regulatory elements from a particular mammalian organism, such as human, will most often function in other mammalian species, such as mouse. Furthermore, in designing recombinant genes that will function across many species, there are consensus sequences for many types of regulatory elements that are known to function across species, e.g., in all mammalian cells, including mouse host cells and human host cells.

As used herein, the expressions “in operable combination,” “in operable order,” “operatively linked,” “operatively joined” and similar phrases, when used in reference to nucleic acids, refer to the operational linkage of nucleic acid sequences placed in functional relationships with each other. For example, an operatively linked promoter, enhancer elements, open reading frame, 5′ and 3′ UTR, and terminator sequences result in the accurate production of an RNA molecule. In some aspects, operatively linked nucleic acid elements result in the transcription of an open reading frame and ultimately the production of a polypeptide (i.e., expression of the open reading frame).

As used herein, the terms “vector,” “vehicle,” “construct” and “plasmid” are used in reference to any recombinant polynucleotide molecule that can be propagated and used to transfer nucleic acid segment(s) from one organism to another. Vectors generally comprise parts which mediate vector propagation and manipulation (e.g., one or more origin of replication, genes imparting drug or antibiotic resistance, a multiple cloning site, operably linked promoter/enhancer elements which enable the expression of a cloned gene, etc.). Vectors are generally recombinant nucleic acid molecules, often derived from bacteriophages, or plant or animal viruses. Plasmids and cosmids refer to two such recombinant vectors. A “cloning vector” or “shuttle vector” or “subcloning vector” contain operably linked parts that facilitate subcloning steps (e.g., a multiple cloning site containing multiple restriction endonuclease target sequences). A nucleic acid vector can be a linear molecule, or in circular form, depending on type of vector or type of application. Some circular nucleic acid vectors can be intentionally linearized prior to delivery into a cell.

As used herein, the term “expression vector” refers to a recombinant vector comprising operably linked polynucleotide elements that facilitate and optimize expression of a desired gene (e.g., a gene that encodes a protein) in a particular host organism (e.g., a bacterial expression vector or mammalian expression vector). Polynucleotide sequences that facilitate gene expression can include, for example, promoters, enhancers, transcription termination sequences, and ribosome binding sites.

As used herein, the term “host cell” refers to any cell that contains a heterologous nucleic acid. The heterologous nucleic acid can be a vector, such as a shuttle vector or an expression vector. In some aspects, the host cell is able to drive the expression of genes that are encoded on the vector. In some aspects, the host cell supports the replication and propagation of the vector. Host cells can be bacterial cells such as E. coli, or mammalian cells (e.g., human cells or mouse cells). When a suitable host cell (such as a suitable mouse cell) is used to create a stably integrated cell line, that cell line can be used to create a complete transgenic organism.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “transformation” and “transfection” mean the introduction of a nucleic acid, e.g., an expression vector, into a recipient cell including introduction of a nucleic acid to the chromosomal DNA of said cell.

By “isolated nucleic acid” or “purified nucleic acid” is meant DNA that is isolated from the naturally-occurring genome of the organism from which the DNA of the invention is derived. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

The term “start of replication” is intended to mean a nucleotide sequence at, which DNA synthesis for replication of the vector begins. Start of replication may occur at one or more points within the vector dependent on the vector being used, such as at one point in a plasmid vector or at several points in an adenovector. The start of replication is generally termed origin of replication (abbreviated ori site) in a plasmid vector.

The term “control sequence” or “control sequences” is intended to mean nucleotide sequences involved in control of a response of action. This includes nucleotide sequences and/or proteins involved in regulating, controlling or affecting the expression of structural genes, or the replication, selection or maintenance of a plasmid or a viral vector. Examples include attenuators, silencers, enhancers, operators, terminators and promoters.

“Exogenous nucleic acids” are nucleic acids which originate outside of the microorganism to which they are introduced. Exogenous nucleic acids may be derived from any appropriate source, including, but not limited to, the microorganism to which they are to be introduced, strains or species of microorganisms which differ from the organism to which they are to be introduced, or they may be artificially or recombinantly created. In one embodiment, the exogenous nucleic acids represent nucleic acid sequences naturally present within the microorganism to which they are to be introduced, and they are introduced to increase expression of or over-express a particular gene (for example, by increasing the copy number of the sequence (for example a gene)). In another embodiment, the exogenous nucleic acids represent nucleic acid sequences not naturally present within the microorganism to which they are to be introduced and allow for the expression of a product not naturally present within the microorganism or increased expression of a gene native to the microorganism (for example in the case of introduction of a regulatory element such as a promoter). The exogenous nucleic acid may be adapted to integrate into the genome of the microorganism to which it is to be introduced or to remain in an extra-chromosomal state.

The term “recombinant microorganism” or “genetically modified microorganism”, as used herein, refers to a microorganism genetically modified or genetically engineered. It means, according to the usual meaning of these terms, that the microorganism of the invention is not found in nature and is modified either by introduction, by deletion or by modification of genetic elements. A microorganism may be modified to express exogenous genes if these genes are introduced into the microorganism with all the elements allowing their expression in the host microorganism. A microorganism may be modified to modulate the expression level of an endogenous gene. The modification or “transformation” of microorganisms with exogenous DNA is a routine task for those skilled in the art.

As used herein, the terms “heterologous” or “exogenous” as applied to polynucleotides or polypeptides refers to molecules that have been rearranged or artificially supplied to a biological system and are not in a native configuration (e.g., with respect to sequence, genomic position or arrangement of parts) or are not native to that particular biological system. These terms indicate that the relevant material originated from a source other than the naturally occurring source, or refers to molecules having a non-natural configuration, genetic location or arrangement of parts. The terms “exogenous” and “heterologous” are sometimes used interchangeably with “recombinant.”

As used herein, the terms “native” or “endogenous” refer to molecules that are found in a naturally occurring biological system, cell, tissue, species or chromosome under study. A “native” or “endogenous” gene is a generally a gene that does not include nucleotide sequences other than nucleotide sequences with which it is normally associated in nature (e.g., a nuclear chromosome, mitochondrial chromosome or chloroplast chromosome). An endogenous gene, transcript or polypeptide is encoded by its natural locus, and is not artificially supplied to the cell.

The nucleic acids disclosed herein may have sequences that vary from the sequences specifically exemplified herein provided they perform substantially the same function. For nucleic acid sequences that encode a protein or peptide this means that the encoded protein or peptide has substantially the same function. For nucleic acid sequences that represent promoter sequences, the variant sequence will have the ability to promote expression of one or more genes. Such nucleic acids may be referred to herein as “functionally equivalent variants”. By way of example, functionally equivalent variants of a nucleic acid include allelic variants, fragments of a gene, genes which include mutations (deletion, insertion, nucleotide substitutions and the like) and/or polymorphisms and the like.

The phrase “functionally equivalent variants” should also be taken to include nucleic acids whose sequence varies as a result of codon optimization for a particular organism. “Functionally equivalent variants” of a nucleic acid herein will preferably have at least approximately 70%, preferably approximately 80%, more preferably approximately 85%, preferably approximately 90%, preferably approximately 95% or greater nucleic acid sequence identity with the nucleic acid identified.

The polypeptides disclosed herein may have sequences that vary from the sequences specifically exemplified herein. These variants may be referred to herein as “functionally equivalent variants”. A functionally equivalent variant of a protein or a peptide includes those proteins or peptides that share at least 40%, preferably 50%, preferably 60%, preferably 70%, preferably 75%, preferably 80%, preferably 85%, preferably 90%, preferably 95% or greater amino acid identity with the protein or peptide identified and has substantially the same function as the peptide or protein of interest. Such variants include within their scope fragments of a protein or peptide wherein the fragment comprises a truncated form of the polypeptide wherein deletions may be from 1 to 5, to 10, to 15, to 20, to 25 amino acids, and may extend from residue 1 through 25 at either terminus of the polypeptide, and wherein deletions may be of any length within the region; or may be at an internal location. Functionally equivalent variants of the specific polypeptides herein should also be taken to include polypeptides expressed by homologous genes in other species of bacteria.

“Substantially the same function” as used herein is intended to mean that the nucleic acid or polypeptide is able to perform the function of the nucleic acid or polypeptide of which it is a variant. One may assess whether a functionally equivalent variant has substantially the same function as the nucleic acid or polypeptide of which it is a variant using any number of known methods.

“Over-express”, “over expression” and like terms and phrases when used in relation to the invention should be taken broadly to include any increase in expression of one or more protein as compared to the expression level of the protein of a parental microorganism under the same conditions. It should not be taken to mean that the protein is expressed at any particular level.

An “appropriate culture medium” designates a medium (e.g., a sterile, liquid media) comprising nutrients essential or beneficial to the maintenance and/or growth of the cell such as carbon sources or carbon substrate, nitrogen sources, for example, peptone, yeast extracts, meat extracts, malt extracts, urea, ammonium sulfate, ammonium chloride, ammonium nitrate and ammonium phosphate; phosphorus sources, for example, monopotassium phosphate or dipotassium phosphate; trace elements (e.g., metal salts), for example magnesium salts, cobalt salts and/or manganese salts; as well as growth factors such as amino acids and vitamins.

General

Disclosed herein is a genetic circuit for L-DOPA biosynthesis containing a positive feedback loop for homeostatic control of L-DOPA production. Production of L-DOPA by 4-hydroxyphenylacetate 3-monooxygenase (HpaB, the product of the hpaB gene, or other enzymes used for L-DOPA production such as tyrosinase or tyrosine hydroxylase activates the L-DOPA responsive transcription factor DopA (SEQ ID NO: 1), which is bound to a specific promoter sequence upstream of the hpaB gene. An exemplary promoter is found in SEQ ID NO: 2. See Wei et al., Genome Engineering Escherichia coli for L-DOPA Overproduction from Glucose, Sci Rep. 2016; 6:30080, July 2016, for a discussion regarding production of L-Dopa from HpaB, herein incorporated by reference in its entirety.

DopA is encoded by gene PP2551 from Pseudomonas putida, for example (SEQ ID NO: 1). Activated DopA recruits bacterial transcriptional machinery to the promoter resulting in increased transcription of the hpaB gene. Increased transcription of the hpaB gene increases the amount of HpaB protein (SEQ ID NO: 3) within the cell, in turn increasing the intracellular level of L-DOPA, resulting in a positive feedback signal. Bacterial cells eventually enter a steady-state phase of L-DOPA production without the need for external induction. HpaC (SEQ ID NO: 4) can also be involved in L-DOPA production, as can be seen in FIG. 3.

According to the above-described design of the expression system, expression of the system can be auto-inducibly and positively feedback-regulated. Such an expression system can be called “the auto-inducible positive feedback regulated expression system”, but for reasons of simplicity, may also be referred to as the expression system.

The novel metabolic pathway described herein is introduced into a host cell using genetic engineering techniques. The term “cell” is meant to include any type of biological cell. The host cell can be a eukaryotic cell or a prokaryotic cell. Preferably, the host cell is a prokaryotic cell such as a bacterial cell; however single cell eukaryotes such as protists or yeasts are also useful as host cells.

Host cells can be individually engineered to express one or more of the pathway enzymes as needed to complete the L-DOPA biosynthetic pathway as described herein; for example, they can be engineered to biosynthesize the starting material tyrosine if they do not natively produce it. Additionally, cells can be engineered to improve uptake of exogenously supplemented L-tyrosine. Preferred host cells are microbial cells, preferably the cells of single-celled microbes such as bacterial cells or yeast cells. Examples of microbial cells that can be engineered to express the L-DOPA biosynthesis pathway as described herein, in addition to E. coli, include a wide variety of bacteria and yeast including but not limited to members of the genera Escherichia, Salmonella, Clostridium, Zymomonas, Pseudomonas, Bacillus, Rhodococcus, Alcaligenes, Klebsiella, Paenibacillus, Lactobacillus, Enterococcus, Arthrobacter, Brevibacterium, Corynebacterium Candida, Hansenula, Pichia and Saccharomyces. Particularly preferred hosts include: Escherichia coli, Bacillus subtilis, Bacillus licheniformis, Alcaligenes eutrophus, Rhodococcus erythropolis, Paenibacillus macerans, Pseudomonas putida, Enterococcus faecium, Saccharomyces cerevisiae, Lactobacillus plantarum, Enterococcus gallinarium and Enterococcus faecalis. In preferred embodiments, the host cell is a bacterial cell, such as an E. coli or Streptomyces caeruleus cell. In a particularly preferred embodiment, the host cell of the present invention is an E. coli cell.

The term “microbe” is used interchangeably with the term “microorganism” and means any microscopic organism existing as a single cell (unicellular), cell clusters, or multicellular relatively complex organisms. Microorganisms include, for example, bacteria, fungi, algae, protozoa, microscopic plants such as green algae, and microscopic animals such as rotifers and planarians. Preferably, a microbial host used in the present invention is single-celled. Notwithstanding the above preferences for bacterial and/or microbial cells, it should be understood the metabolic pathway of the invention can be introduced without limitation into the cell of an animal, plant, insect, yeast, protozoan, bacterium, or archaebacterium.

A cell that has been genetically engineered to express one or more enzyme(s) described herein for L-DOPA biosynthesis may be referred to as a “host” cell, a “recombinant” cell, a “metabolically engineered” cell, a “genetically engineered” cell or simply an “engineered” cell. These and similar terms are used interchangeably. A genetically engineered cell contains one or more artificial sequences of nucleotides which have been created through standard molecular cloning techniques to bring together genetic material that is not natively found together. DNA sequences used in the construction of recombinant DNA molecules can originate from any species.

Alternatively, DNA sequences that do not occur anywhere in nature may be created by the chemical synthesis of DNA, and incorporated into recombinant molecules. Proteins that result from the expression of recombinant DNA are often termed recombinant proteins. Examples of recombination are described in more detail below and may include inserting foreign polynucleotides (obtained from another species of cell) into a cell, inserting synthetic polynucleotides into a cell, or relocating or rearranging polynucleotides within a cell. Any form of recombination may be considered to be genetic engineering and therefore any recombinant cell may also be considered to be a genetically engineered cell.

Genetically engineered cells are also referred to as “metabolically engineered” cells when the genetic engineering modifies or alters one or more particular metabolic pathways so as to cause a change in metabolism. The goal of metabolic engineering is to improve the rate and conversion of a substrate into a desired product. General laboratory methods for introducing and expressing or overexpressing native and nonnative proteins such as enzymes in many different cell types (including bacteria, plants, and animals) are routine and well known in the art; see, e.g., Sambrook et al, Molecular Cloning: A Laboratory Manual, Cold Spring Harbor Laboratory Press (1989), and Methods for General and Molecular Bacteriology, (eds. Gerhardt et al.) American Society for Microbiology, chapters 13-14 and 16-18 (1994).

The introduction of the novel biosynthetic pathway of the invention into a cell involves expression or overexpression of one or more enzymes included in the novel pathway. An enzyme is “overexpressed” in a recombinant cell when the enzyme is expressed at a level higher than the level at which it is expressed in a comparable wild-type cell. In cells that do not express a particular endogenous enzyme, or in cells in which the enzyme is not endogenous (i.e., the enzyme is not native to the cell), any level of expression of that enzyme in the cell is deemed an “overexpression” of that enzyme for purposes of the present invention.

As will be appreciated by a person of skill in the art, overexpression of an enzyme can be achieved through a number of molecular biology techniques. For example, overexpression can be achieved by introducing into the host cell one or more copies of a polynucleotide encoding the desired enzyme. The polynucleotide encoding the desired enzyme may be endogenous or heterologous to the host cell. Preferably, the polynucleotide is introduced into the cell using a vector; however, naked DNA may also be used. The polynucleotide may be circular or linear, single-stranded or double stranded, and can be DNA, RNA, or any modification or combination thereof. The vector can be any molecule that may be used as a vehicle to transfer genetic material into a cell. Examples of vectors include plasmids, viral vectors, cosmids, and artificial chromosomes, without limitation. Examples of molecular biology techniques used to transfer nucleotide sequences into a microorganism include, without limitation, transfection, electroporation, transduction, and transformation. These methods are well known in the art. Insertion of a vector into a target cell is usually called transformation for bacterial cells and transfection for eukaryotic cells, however insertion of a viral vector is often called transduction. The terms transformation, transfection, and transduction, for the purpose of the instant invention, are used interchangeably herein. A polynucleotide which has been transferred into a cell via the use of a vector is often referred to as a transgene.

Preferably, the vector is an expression vector. An “expression vector” or “expression construct” is any vector that is used to introduce a specific polynucleotide into a target cell such that once the expression vector is inside the cell, the protein that is encoded by the polynucleotide is produced by the cellular transcription and translation machinery. Typically an expression vector includes regulatory sequences operably linked to the polynucleotide encoding the desired enzyme. Regulatory sequences are common to the person of the skill in the art and may include for example, an origin of replication, a promoter sequence, and/or an enhancer sequence. The polynucleotide encoding the desired enzyme can exist extrachromosomally or can be integrated into the host cell chromosomal DNA. Extrachromosomal DNA may be contained in cytoplasmic organelles, such as mitochondria (in most eukaryotes), and in chloroplasts and plastids (in plants). More typically, extrachromosomal DNA is maintained within the vector on which it was introduced into the host cell. In many instances, it may be beneficial to select a high copy number vector in order to maximize the expression of the enzyme. Optionally, the vector may further contain a selectable marker. Certain selectable markers may be used to confirm that the vector is present within the target cell. Other selectable markers may be used to further confirm that the vector and/or transgene has integrated into the host cell chromosomal DNA. The use of selectable markers is common in the art and the skilled person would understand and appreciate the many uses of selectable markers.

The genetically engineered cell of the invention expresses or overexpresses L-DOPA. Where a cell does not express HpaB/C endogenously, any expression of HpaB/C is considered to be “overexpression.” Determination of whether HpaB/C is expressed or overexpressed can easily be made by a person of skill in the art using a basic in vitro or in vivo enzyme assays. Common methods for measuring the amount of the product may include, without limitation, chromatographic techniques such as size exclusion chromatography, separation based on charge or hydrophobicity, ion exchange chromatography, affinity chromatography, or liquid chromatography. The genetically engineered cell of the invention will yield a greater activity than a wild-type cell in such an assay. Additionally, or alternatively, the amount of HpaB/C can be quantified and compared by obtaining protein extracts from the genetically engineered cell and a comparable wild-type cell and subjecting the extracts to any of number of protein quantification techniques which are well known in the art. Methods of protein quantification may include, without limitation, SDS-PAGE in combination with western blotting and mass spectrometry.

A gene encoding DopA may be obtained from a suitable biological source, such as a bacterial cell, using standard molecular cloning techniques, or techniques known in the art for synthesizing nucleic acid. For example, genes may be isolated using polymerase chain reaction (PCR) using primers designed by standard primer design software which is commonly used in the art. The cloned sequences are easily ligated into any standard expression vector by the skilled person.

In addition to overexpressing HpaB, the genetically engineered cell of the invention also expresses DopA. This comparison is likewise easily made by a person of skill in the art using a basic in vitro or in vivo enzyme assays. Briefly, DopA activity can be measured and compared by obtaining crude enzyme extracts from a genetically engineered cell and a comparable wild-type cell, subjecting a suitable substrate to each enzyme extract, and measuring the amount of product (i.e., L-DOPA). Common methods for measuring the amount of the product and common methods of protein quantification are well known in the art and are listed in brief above.

Any protein which functions as a specific L-DOPA responsive transcriptional activor can be utilized in the metabolic pathway of the invention. Preferably, the protein possessing DopA functionality is soluble and not membrane-associated, allowing it to be expressed and active in a cytosolic environment such as inside a bacterial cell. Any biological source of DopA functionality can be utilized. Examples of biological sources of DopA include gene PP2551 from Pseudomonas putida (SEQ ID NO: 1).

In one embodiment of the genetically engineered cell, separate, independent expression vectors are introduced into the host cell. A first expression vector is used to express HpaB and/or HpaC, and a second expression vector can be used to express DopA. In another embodiment, a single vector may be engineered to express both HpaB/C and DopA, as well as the associated promoter disclosed herein (SEQ ID NO: 2). When a single expression vector is used, each nucleotide sequence encoding a desired enzyme may be under the control of a single regulatory sequence or, alternatively, each nucleotide sequence encoding a desired enzyme may be under the control of independent regulatory sequences. An exemplary expression system can be seen in FIG. 3.

The expression system disclosed herein can also be modified in a number of other ways in order to maximize efficiency of the system, and yield of L-DOPA. Examples include, but are not limited to, deletion of transcriptional regulator tyrosine repressor (tyrR), deletion of transcriptional regulator carbon storage regulator A (csrA); alteration of the glucose transport system of the bacterium from phosphotransferase system (PTS) to ATP-dependent uptake; alteration of the phosphorylation system of the bacterium to overexpress galactose permease gene (galP) and glucokinase gene (glk); knock-outs of glucose-6-phosphate dehydrogenase gene (zwj) and prephenate dehydratase and its leader peptide genes (pheLA); and integration of a fusion protein chimera of a downstream pathway of chorismate.

The present invention further provides a method for producing L-DOPA, as well as L-DOPA derivatives and downstream metabolites, using the genetically engineered cell described herein. Briefly, and as described and illustrated in more detail elsewhere herein, the host cell is engineered to contain a novel biosynthetic pathway. Specifically, the host cell is engineered to overexpress HpaB and HpaC. The host cell is further engineered to overexpress DopA, as activated DopA recruits bacterial transcriptional machinery to the promoter resulting in increased transcription of the hpaB gene. Increased transcription of the hpaB gene increases the amount of HpaB protein within the cell, in turn increasing the intracellular level of L-DOPA, resulting in a positive feedback signal.

The L-DOPA produced via the novel biosynthetic pathway can be isolated and optionally purified from any genetically engineered cell described herein. It can be isolated directly from the cells, or from the culture medium, for example, during an aerobic or anaerobic fermentation process. Isolation and/or purification can be accomplished using known methods. The present invention may also be extended by introducing additional selected metabolic enzymes to permit the microbial synthesis, production, isolation and/or purification of many other compounds derived from L-DOPA.

The genetically engineered cells of the invention can be cultured aerobically or anaerobically, or in a multiple phase fermentation that makes use of periods of anaerobic and aerobic fermentation. Preferably, the cells are cultured aerobically. Batch fermentation, continuous fermentation, or any other fermentation method may be used.

Importantly, the present invention permits a “total synthesis” or “de novo” biosynthesis of L-DOPA in the genetically engineered cell. In other words, it is not necessary to supply the genetically engineered cells with precursors or intermediates; L-DOPA can be produced in a steady-state using ordinary inexpensive carbon sources such as glucose, glycerol, gluconate, acetate and the like.

Disclosed herein are various amino acid and nucleic acid sequences. Contemplated herein are variants of these sequences. As used herein, the expression “variant” refers to a first composition (e.g., a first molecule), that is related to a second composition (e.g., a second molecule, also termed a “parent” molecule). The variant molecule can be derived from, isolated from, based on or homologous to the parent molecule The term variant can be used to describe either polynucleotides or polypeptides.

As applied to polynucleotides, a variant molecule can have entire nucleotide sequence identity with the original parent molecule, or alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99% or more identical in nucleotide sequence compare to the original nucleotide sequence. Polynucleotide variants also include polynucleotides comprising the entire parent polynucleotide, and further comprising additional fused nucleotide sequences. Polynucleotide variants also includes polynucleotides that are portions or subsequences of the parent polynucleotide, for example, unique subsequences (e.g., as determined by standard sequence comparison and alignment techniques) of the polynucleotides disclosed herein are also encompassed by the invention.

In another aspect, polynucleotide variants includes nucleotide sequences that contain minor, trivial or inconsequential changes to the parent nucleotide sequence. For example, minor, trivial or inconsequential changes include changes to nucleotide sequence that (i) do not change the amino acid sequence of the corresponding polypeptide, (ii) occur outside the protein-coding open reading frame of a polynucleotide, (iii) result in deletions or insertions that may impact the corresponding amino acid sequence, but have little or no impact on the biological activity of the polypeptide, (iv) the nucleotide changes result in the substitution of an amino acid with a chemically similar amino acid. In the case where a polynucleotide does not encode for a protein (for example, the promoter, as disclosed in SEQ ID NO: 2), variants of that polynucleotide can include nucleotide changes that do not result in loss of function of the polynucleotide. In another aspect, conservative variants of the disclosed nucleotide sequences that yield functionally identical nucleotide sequences are encompassed by the invention. One of skill will appreciate that many variants of the disclosed nucleotide sequences are encompassed by the invention.

Variant polypeptides are also disclosed. As applied to proteins, a variant polypeptide can have entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence.

Polypeptide variants include polypeptides comprising the entire parent polypeptide, and further comprising additional fused amino acid sequences. Polypeptide variants also includes polypeptides that are portions or subsequences of the parent polypeptide, for example, unique subsequences (e.g., as determined by standard sequence comparison and alignment techniques) of the polypeptides disclosed herein are also encompassed by the invention.

In another aspect, polypeptide variants includes polypeptides that contain minor, trivial or inconsequential changes to the parent amino acid sequence. For example, minor, trivial or inconsequential changes include amino acid changes (including substitutions, deletions and insertions) that have little or no impact on the biological activity of the polypeptide, and yield functionally identical polypeptides, including additions of non-functional peptide sequence. In other aspects, the variant polypeptides of the invention change the biological activity of the parent molecule. One of skill will appreciate that many variants of the disclosed polypeptides are encompassed by the invention.

In some aspects, polynucleotide or polypeptide variants of the invention can include variant molecules that alter, add or delete a small percentage of the nucleotide or amino acid positions, for example, typically less than about 10%, less than about 5%, less than 4%, less than 2% or less than 1%.

As used herein, the term “conservative substitutions” in a nucleotide or amino acid sequence refers to changes in the nucleotide sequence that either (i) do not result in any corresponding change in the amino acid sequence due to the redundancy of the triplet codon code, or (ii) result in a substitution of the original parent amino acid with an amino acid having a chemically similar structure. Conservative substitution tables providing functionally similar amino acids are well known in the art, where one amino acid residue is substituted for another amino acid residue having similar chemical properties (e.g., aromatic side chains or positively charged side chains), and therefore does not substantially change the functional properties of the resulting polypeptide molecule.

The following are groupings of natural amino acids that contain similar chemical properties, where substitutions within a group is a “conservative” amino acid substitution. This grouping indicated below is not rigid, as these natural amino acids can be placed in different grouping when different functional properties are considered Amino acids having nonpolar and/or aliphatic side chains include: glycine, alanine, valine, leucine, isoleucine and proline Amino acids having polar, uncharged side chains include: serine, threonine, cysteine, methionine, asparagine and glutamine.

Amino acids having aromatic side chains include: phenylalanine, tyrosine and tryptophan Amino acids having positively charged side chains include: lysine, arginine and histidine Amino acids having negatively charged side chains include: aspartate and glutamate.

As used herein, the terms “identical” or “percent identity” in the context of two or more nucleic acids or polypeptides refer to two or more sequences or subsequences that are the same (“identical”) or have a specified percentage of amino acid residues or nucleotides that are identical (“percent identity”) when compared and aligned for maximum correspondence with a second molecule, as measured using a sequence comparison algorithm (e.g., by a BLAST alignment, or any other algorithm known to persons of skill), or alternatively, by visual inspection.

The phrase “substantially identical,” in the context of two nucleic acids or polypeptides refers to two or more sequences or subsequences that have at least about 60%, about 80%, about 90%, about 90-95%, about 95%, about 98%, about 99% or more nucleotide or amino acid residue identity, when compared and aligned for maximum correspondence using a sequence comparison algorithm or by visual inspection. Such “substantially identical” sequences are typically considered to be “homologous,” without reference to actual ancestry. Preferably, the “substantial identity” between nucleotides exists over a region of the polynucleotide at least about 50 nucleotides in length, at least about 100 nucleotides in length, at least about 200 nucleotides in length, at least about 300 nucleotides in length, or at least about 500 nucleotides in length, most preferably over their entire length of the polynucleotide. Preferably, the “substantial identity” between polypeptides exists over a region of the polypeptide at least about 50 amino acid residues in length, more preferably over a region of at least about 100 amino acid residues, and most preferably, the sequences are substantially identical over their entire length.

The phrase “sequence similarity,” in the context of two polypeptides refers to the extent of relatedness between two or more sequences or subsequences. Such sequences will typically have some degree of amino acid sequence identity, and in addition, where there exists amino acid non-identity, there is some percentage of substitutions within groups of functionally related amino acids. For example, substitution (misalignment) of a serine with a threonine in a polypeptide is sequence similarity (but not identity).

As used herein, the term “homologous” refers to two or more amino acid sequences when they are derived, naturally or artificially, from a common ancestral protein or amino acid sequence. Similarly, nucleotide sequences are homologous when they are derived, naturally or artificially, from a common ancestral nucleic acid. Homology in proteins is generally inferred from amino acid sequence identity and sequence similarity between two or more proteins. The precise percentage of identity and/or similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95%, or 99% or more, can also be used to establish homology. Methods for determining sequence similarity percentages (e.g., BLASTP and BLASTN using default parameters) are generally available.

As used herein, the terms “portion,” “subsequence,” “segment” or “fragment” or similar terms refer to any portion of a larger sequence (e.g., a nucleotide subsequence or an amino acid subsequence) that is smaller than the complete sequence from which it was derived. The minimum length of a subsequence is generally not limited, except that a minimum length may be useful in view of its intended function. The subsequence can be derived from any portion of the parent molecule. In some aspects, the portion or subsequence retains a critical feature or biological activity of the larger molecule, or corresponds to a particular functional domain of the parent molecule, for example, the DNA-binding domain, or the transcriptional activation domain. Portions of polynucleotides can be any length, for example, at least 5, 10, 15, 20, 25, 30, 40, 50, 75, 100, 150, 200, 300 or 500 or more nucleotides in length.

As used herein, the term “kit” is used in reference to a combination of articles that facilitate a process, method, assay, analysis or manipulation of a sample. Kits can contain written instructions describing how to use the kit (e.g., instructions describing the methods of the present invention), chemical reagents or enzymes required for the method, primers and probes, as well as any other components.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

SEQUENCES

Seq ID No. 1-Amino acid sequence of DopA.
MNPTFASLSLAHLRTLDHLLQLKNLSHAAERLGVSQSALSRQLAHLREA
FDDPLLVRQGRGYVLSEHAEALVEPLRQVLEELHALRQPAIFDPARCER
RFCLAASDYVAEHMLPLLVAALEREAPGVSLEYRTWQAGQYALLASGEI
DLATTLFDESPPNLHGRLLGEDRAVCLMRQDHPLAAQAALSQADYLAYK
HVRISGGGDKDSFIDRHLRAQGLQRRVSLEVPFFCATVQVIASSQAVAT
VPEHIARQLSRLHDLAWRPLGFIDHSQRYWVVWHQRLQASAEHRWLRNR
VFELWRQSQFGVQGGHAGSP*
Seq ID No. 2-Minimal promoter sequence for
activation by DopA.
catagcagctatgcggtaagcgaggttattcggctggggataggtgcct
agactggggcattgtgttgattgtgcggcttcttcgcggctgtaggcgc
gggtttacccgcgaaagggccagcacaggcaatggataacccTAAGGAG
GtacgtaATG
Seq ID No. 3-Amino acid sequence of HpaB.
MKPEDFRASTQRPFTGEEYLKSLQDGREIYIYGERVKDVTTHPAFRNAA
ASVAQLYDALHKPEMQDSLCWNTDTGSGGYTHKFFRVAKSADDLRQQRD
AIAEWSRLSYGWMGRTPDYKAAFGCALGANPGFYGQFEQNARNWYTRIQ
ETGLYFNHAIVNPPIDRHLPTDKVKDVYIKLEKETDAGIIVSGAKVVAT
NSALTHYNMIGFGSAQVMGENPDFALMFVAPMDADGVKLISRASYEMVA
GATGSPYDYPLSSRFDENDAILVMDNVLIPWENVLIYRDFDRCRRWTME
GGFARMYPLQACVRLAVKLDFITALLKKSLECTGTLEFRGVQADLGEVV
AWRNTFWALSDSMCSEATPWVNGAYLPDHAALQTYRVLAPMAYAKIKNI
IERNVTSGLIYLPSSARDLNNPQIDQYLAKYVRGSNGMDHVQRIKILKL
MWDAIGSEFGGRHELYEINYSGSQDEIRLQCLRQAQSSGNMDKMMAMVD
RCLSEYDQNGWTVPHLHNNDDINMLDKLLK*
Seq ID No. 4-Amino acid sequence of HpaC.
MQLDEQRLRFRDAMASLSAAVNIITTEGDAGQCGITATAVCSVTDTPPS
LMVCINANSAMNPVFQGNGKLCVNVLNHEQELMARHFAGMTGMAMEERF
SLSCWQKGPLAQPVLKGSLASLEGEIRDVQAIGTHLVYLVEIKNIILSA
EGHGLIYFKRRFHPVMLEMEAAI*

1. A genetically engineered cell capable of producing L-3,4-dihydroxyphenylalanine (L-DOPA), wherein said cell comprises a gene encoding PP2551 of Pseudomonas putida.

2. The cell of claim 1, wherein an amino acid sequence encoded by PP2251 of Pseudomonas putida comprises SEQ ID NO: 1.

3. The cell of claim 1, further comprising a promoter recognized by PP2251.

4. The cell of claim 3, wherein the promoter recognized by PP2251 comprises SEQ ID NO: 2.

5. The cell of claim 1, wherein the cell further comprises genes hpaB and hpaC encoding HpaB and HpaC respectively.

6. The cell of claim 5, wherein the amino acid sequence encoded by hpaB is SEQ ID NO: 3.

7. The cell of claim 5, wherein the amino acid sequence encoded by hpaC is SEQ ID NO: 4.

8. The cell of claim 1, wherein the cell is capable of producing L-DOPA at a steady state.

9. The cell of claim 1, wherein transcriptional regulator tyrosine repressor (tyrR) has been deleted.

10. The cell of claim 1, wherein transcriptional regulator carbon storage regulator A (csrA) has been deleted.

11. The cell of claim 1, wherein glucose transport system of the bacterium has been altered from phosphotransferase system (PTS) to ATP-dependent uptake.

12. The cell of claim 1, wherein phosphorylation system of the cell has been altered to overexpress galactose permease gene (galP) and glucokinase gene (glk).

13. The cell of claim 1, wherein glucose-6-phosphate dehydrogenase gene (zwf) and prephenate dehydratase and its leader peptide genes (pheLA) have been knocked out.

14. The cell of claim 1, wherein a fusion protein chimera of a downstream pathway of chorismate has been integrated.

15. A plasmid comprising a gene encoding PP2551 of Pseudomonas putida, a promoter thereof, and genes encoding hpaB and hpaC.

16. A cell line comprising the plasmid of claim 16.

17. A method of producing L-DOPA, comprising transforming a cell with a gene encoding PP2551 of Pseudomonas putida.

18. The method of claim 17, wherein an amino acid sequence encoded by PP2251 of Pseudomonas putida comprises SEQ ID NO: 1.

19. The method of claim 17, further comprising a promoter recognized by PP2251.

20. The method of claim 19, wherein the promoter recognized by PP2251 comprises SEQ ID NO: 2.

21. (canceled)

22. (canceled)

23. (canceled)


SEQUENCE LISTING (4 sequences)

SEQ ID NO: 1 - protein, 314 aa, Pseudomonas putida
MNPTFASLSLAHLRTLDHLLQLKNLSHAAERLGVSQSALSRQLAHLREAFDDPLLVRQGRGYVLSEHAEALVEPLRQVLEELHALRQPAIFDPARCERRFCLAASDYVAEHMLPLLVAALEREAPGVSLEYRTWQAGQYALLASGEIDLATTLFDESPPNLHGRLLGEDRAVCLMRQDHPLAAQAALSQADYLAYKHVRISGGGDKDSFIDRHLRAQGLQRRVSLEVPFFCATVQVIASSQAVATVPEHIARQLSRLHDLAWRPLGFIDHSQRYWVVWHQRLQASAEHRWLRNRVFELWRQSQFGVQGGHAGSP

SEQ ID NO: 2 - DNA, 157 bp, homo sapiens
catagcagctatgcggtaagcgaggttattcggctggggataggtgcctagactggggcattgtgttgattgtgcggcttcttcgcggctgtaggcgcgggtttacccgcgaaagggccagcacaggcaatggataaccctaaggaggtacgtaatg

SEQ ID NO: 3 - protein, 520 aa, Homo sapiens
MKPEDFRASTQRPFTGEEYLKSLQDGREIYIYGERVKDVTTHPAFRNAAASVAQLYDALHKPEMQDSLCWNTDTGSGGYTHKFFRVAKSADDLRQQRDAIAEWSRLSYGWMGRTPDYKAAFGCALGANPGFYGQFEQNARNWYTRIQETGLYFNHAIVNPPIDRHLPTDKVKDVYIKLEKETDAGIIVSGAKVVATNSALTHYNMIGFGSAQVMGENPDFALMFVAPMDADGVKLISRASYEMVAGATGSPYDYPLSSRFDENDAILVMDNVLIPWENVLIYRDFDRCRRWTMEGGFARMYPLQACVRLAVKLDFITALLKKSLECTGTLEFRGVQADLGEVVAWRNTFWALSDSMCSEATPWVNGAYLPDHAALQTYRVLAPMAYAKIKNIIERNVTSGLIYLPSSARDLNNPQIDQYLAKYVRGSNGMDHVQRIKILKLMWDAIGSEFGGRHELYEINYSGSQDEIRLQCLRQAQSSGNMDKMMAMVDRCLSEYDQNGWTVPHLHNNDDINMLDKLLK

SEQ ID NO: 4 - protein, 170 aa, homo sapiens
MQLDEQRLRFRDAMASLSAAVNIITTEGDAGQCGITATAVCSVTDTPPSLMVCINANSAMNPVFQGNGKLCVNVLNHEQELMARHFAGMTGMAMEERFSLSCWQKGPLAQPVLKGSLASLEGEIRDVQAIGTHLVYLVEIKNIILSAEGHGLIYFKRRFHPVMLEMEAAI